(19) United States
(12) Patent Application Publication
(10) Pub. No.: US 2002/0006448 A1
(43) Pub. Date: Jan. 17, 2002

(54) Compositions and methods for treating back and leg discomfort

(76) Inventor: Yuanjin Tao, Fremont, CA (US)

Correspondence Address: Gladys H. Monroy, Morrison & Foerster LLP, 755 Page Mill Road, Palo Alto, CA, 94304-1018, US

(21) Appl. No.: 09/872,609

(22) Filed: Jun. 1, 2001

Related U.S. Application Data
(60) Provisional application No. 60/208,793, filed on Jun. 1, 2000.

(51) Int. Cl. A61K 35/78

(52) U.S. Cl. 424/739; 424/764; 424/725; 424/763

(57) ABSTRACT

Compositions and methods for treating back or leg discomfort are provided. The compositions comprise singly or in combination herbals, natural nutritional supplements, minerals and vitamins. Methods of making these compositions are also provided.

### CROSS-REFERENCE TO RELATED APPLICATIONS

This application claims the benefit of U.S. Provisional Application Ser. No. 60/208,793, filed Jun. 1, 2000, hereby incorporated in its entirety by reference.

### TECHNICAL FIELD

This invention relates to compositions and methods for treating back and leg disorders. More specifically, it relates to nutraceutical compositions for treating back or leg discomfort symptoms, and methods for treating these symptoms with said compositions.

### BACKGROUND ART

Back pain is the most prevalent medical disorder in industrialized societies, and the second leading symptom prompting all physician visits in the United States. Deyo, R. A. and Phillips, W. R., Spine (1996), 21: 2826-2832. Low back pain disables a significant number of Americans and remains an expensive health problem that costs the country an estimated $24 billion (in 1990) in medical expenses annually. Borenstein, D., Curr. Opin. Rheumatol (1992), 4(2): 226-32. Because of the physical alignment and connection between the back and the legs, leg pain is often associated with back pain.

A nutraceutical composition for treating back and leg disorders is known (Capolla Back and Leg Factors, Amino Foods Technologies, San Jose, Calif.). The ingredients in this formula include angelica, yanhusuo, devil's claw, black cohosh, vitamin B complex, ascorbic acid, natural vitamin E and magnesium.

A double health wine prepared by soaking Chinese medicinal materials in spirit for treating back and leg pain, and psiatic strain, is known. Chinese Unexamined Patent Application No. 1216254. The medicinal materials include giant knotweed, herba rubi parvifolii and radix clematidis.

In view of the seriousness and prevalence of back or leg discomfort symptoms, and the corresponding need to alleviate said symptoms without significant side effects, it would be advantageous to have compositions and methods that are efficacious and safe to address this need. The invention described and claimed in this specification presents such compositions and methods.

All references cited herein, including patent applications and publications, are incorporated by reference in their entirety.

### DISCLOSURE OF THE INVENTION

Compositions and methods for treating back or leg discomfort are provided. The compositions comprise substances in quantities that are effective in relieving back or leg discomfort. Methods of treating back or leg discomfort comprising administration of the claimed compositions are also provided. Methods of making compositions of the invention are also provided.

Accordingly, in one aspect, the invention provides a composition for treating back or leg discomfort comprising 1 substance selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In another aspect, the invention provides a composition for treating back or leg discomfort comprising 2 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In another aspect, the invention provides a composition for treating back or leg discomfort comprising 3 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In another aspect, the invention provides a composition for treating back or leg discomfort comprising 4 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In another aspect, the invention provides a composition for treating back or leg discomfort comprising 5 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In another aspect, the invention provides a composition for treating back or leg discomfort comprising a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

In one embodiment, the invention provides a composition selected from the group consisting of the compositions of the aspects of the invention described in the preceding paragraphs, further comprising a substance selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium. Thus, for example, a composition may comprise 1 or 2 substances selected from the group consisting of a member of the botanical group Millettia and a member of the botanical group Siegesbeckia, further comprising a member of the botanical group Angelica pubescens.

In one embodiment, the invention provides a composition selected from the group consisting of the compositions of the aspects of the invention described in the preceding paragraphs, further comprising 2 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 3 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 4 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 5 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 6 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 7 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 8 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 9 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 10 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 11 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 12 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising 13 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In one embodiment, the invention provides a composition selected from the group consisting of the compositions of the aspects of the invention described in the preceding paragraphs, further comprising a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

In various embodiments of the compositions of the invention described in the preceding and following paragraphs, the member of the botanical group Eucommia may be duzhong, the member of the botanical group Achyranthes may be huainiuxi, the member of the botanical group Millettia may be jixueteng, the member of the botanical group Homalomena may be qiannianjian, the member of the botanical group Cinnamomum cassia may be rougui, and/or the member of the botanical group Siegesbeckia may be xiquancao. In some embodiments, the member of the botanical group Eucommia may be duzhong, the member of the botanical group Achyranthes may be huainiuxi, the member of the botanical group Millettia may be jixueteng, the member of the botanical group Homalomena may be qiannianjian, the member of the botanical group Cinnamomum cassia may be rougui, the member of the botanical group Siegesbeckia may be xiquancao, the member of the botanical group Rosa may be jinyingzi, the member of the botanical group Speranskia may be tougucao, the member of the botanical group Angelica pubescens may be duhuo, the member of the botanical group Clematis may be weilingxian, the member of the botanical group Lycopodium may be shenjincao, the member of the botanical group Harpagophytum may be devil's claw, and/or the member of the botanical group Cimicifuga may be black cohosh.

Accordingly, in one aspect, the invention provides a composition for treating back or leg discomfort comprising a substance selected from the group consisting of duzhong, huainiuxi, jixueteng, qiannianjian, rougui and xiquancao.

In another aspect, the invention provides a composition for treating back or leg discomfort comprising 2 substances selected from the group consisting of duzhong, huainiuxi, jixueteng, qiannianjian, rougui and xiquancao.

In another aspect, the invention provides a composition for treating back or leg discomfort comprising 3 substances selected from the group consisting of duzhong, huainiuxi, jixueteng, qiannianjian, rougui and xiquancao.

In another aspect, the invention provides a composition for treating back or leg discomfort comprising 4 substances selected from the group consisting of duzhong, huainiuxi, jixueteng, qiannianjian, rougui and xiquancao.

In another aspect, the invention provides a composition for treating back or leg discomfort comprising 5 substances selected from the group consisting of duzhong, huainiuxi, jixueteng, qiannianjian, rougui and xiquancao.

In yet another aspect, the invention provides a composition for treating back or leg discomfort comprising duzhong, huainiuxi, jixueteng, qiannianjian, rougui and xiquancao.

In one embodiment, the invention provides a composition selected from the group consisting of the compositions of the aspects of the invention described in the preceding paragraphs, further comprising a substance selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 2 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 3 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 4 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 5 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 6 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 7 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 8 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 9 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 10 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 11 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 12 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising 13 substances selected from the group consisting of jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In another embodiment, the invention provides a composition selected from the group consisting of the compositions of the aspects of the invention described in the preceding paragraphs, further comprising jinyingzi, tougucao, duhuo, weilingxian, shenjincao, vitamin B-1, vitamin B-2, vitamin B-6, vitamin B-12, vitamin C, vitamin E, devil's claw, black cohosh and selenium.

In one aspect, the invention provides a method of treating back or leg discomfort comprising administering to the individual an effective amount of a composition selected from the group consisting of the compositions of aspects and embodiments of the invention described in the preceding paragraphs, whereby back or leg discomfort is treated.

In another aspect, the invention provides method of making a composition for treating back or leg discomfort, said method comprising combining at least two (preferably 2, 3, 4, 5 or 6) substances (preferably in an effective amount) selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia. In some embodiments of these methods, said at least two substances are combined with at least one (preferably 1, 2, 3, 4, 5, 6, 7, 8, 9, 10, 11, 12, 13, or 14) substance (preferably in an effective amount) selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium. In some embodiments, said combining is by mixing (such as by stirring, agitation or vibration). In some embodiments, the substances are packaged in the form of capsules, preferably in size “0”, “00”, “000 ”, “2”, “3” or “4.” In yet other embodiments, the substances are combined in powder form, preferably to at least 30%, 60%, or 90% mixture consistency, or to homogeneity.

### MODES FOR CARRYING OUT THE INVENTION

The present invention discloses compositions comprising single or multiple substances that are effective and safe for treating symptoms of back or leg discomfort. The invention further provides methods of treating back or leg discomfort using the disclosed compositions. The invention also provides methods of making compositions of the invention.

Definitions

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the compositions of the present invention.

An “effective amount” is an amount of a composition or substance(s) sufficient to effect beneficial or desired results in the treatment of back or leg discomfort after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Back or leg discomfort,” as used herein, refers to symptoms involving the back or leg that prevent the normal functioning or use of the back or leg. These symptoms can be those presently indicated as manifestations or any condition that an individual finds to be non-conducive to the optimal or normal use of the back or leg. Such symptoms include, but are not limited to, pain, soreness, stiffness and/or general discomfort of the back or leg area. They include symptoms due to abnormalities in muscles, ligaments, nerves, bones and/or joints in the back or leg area. Symptoms can also be the result of degenerative or inflammatory diseases, tissue wear and tear, injury, tumors, infections and/or congenital abnormalities that are known, believed, or thought to affect the normal or comfortable functioning of the back or leg.

“Individual,” as used herein, refers to a vertebrate, preferably a mammal, more preferably a human.

A “botanical group,” as used herein, refers to a group of botanical entities that are capable of providing similar physiological effect(s) in the compositions of the invention. These botanical entities may or may not belong to the same botanical classification (such as genus, family). “Duzhong,” as used herein, refers to extracts of duzhong. It is also known as cotex eucommiae ulmoidis and eucommia ulmoides Oliv. A member of the botanical group Eucommia is a substance that is capable of providing a similar physiological effect(s) as that provided by duzhong in the compositions of the invention, and is preferably selected from a group comprising Cotex eucommiae ulmoidis; Eucommia ulmoides Oliv.

“Huainiuxi,” as used herein, refers to extracts of huainiuxi. It is also known as radix achyranthis bidentatae and achyranthes bidentata B1. A member of the botanical group Achyranthes is a substance that is capable of providing a similar physiological effect(s) as that provided by huainiuxi in the compositions of the invention, and is preferably selected from a group comprising Radix achyranthis bidentatae; Achyranthes bidentata Blume; Achyranthes longifolia Makino; Cyathula capitata Moq.; Cyathula officinalis Kuan.

“Jixueteng,” as used herein, refers to extracts of jixueteng. It is also known as radix et caulis jixueteng and millettia dielsiana Harms. A member of the botanical group Millettia is a substance that is capable of providing a similar physiological effect(s) as that provided by jixueteng in the compositions of the invention, and is preferably selected from a group comprising Radix et caulis jixueteng; Millettia dielsiana Harms.; Millettia dielsiana Harms. Ex Diels.; Millettia dielsiana; Millettia reticulata benth.; Millettia nitida Benth; Millettia cinerea; Milletta dielsiana; Callerya cinerea; Mucuna sempervirens Hemsl.; Spatholobus suberectus Dunn.; Spatholobus harmandii Gagnep; Sargentodoxa cuneata (Oliv.) Benth. et Wils.

“Qiannianjian,” as used herein, refers to extracts of qiannianjian. It is also known as rhizoma homalomenae occultae and homalomena occulta Schott. A member of the botanical group Homalomena is a substance that is capable of providing a similar physiological effect(s) as that provided by qiannianjian in the compositions of the invention, and is preferably selected from a group comprising Rhizoma homalomenae occultae; Homalomena occulta Schott; Homalomena occulta(Lour.) Schott [H.tonkinensisEngl.]

“Rougui,” as used herein, refers to extracts of rougui. It is also known as cortex cinnamomi cassiae and Cinnamomum cassia Presl. A member of the botanical group Cinnamomum cassia is a substance that is capable of providing a similar physiological effect(s) as that provided by rougui in the compositions of the invention, and is preferably selected from a group comprising Ramulus cinnamomi cassiae; Cinnamomum cassia Blume; Cinnamomum cassia Presl.; Cinnamomum loureiri Ness.; Cinnamomum zeylanicum Blume; Cinnamomum argenteum Gamble.; Cinnamomum tamala Nees. et. Eberm; Cinnamomum wilsonii Gamble; Cinnamomum japonicum Sieb; Cinnamomum pedunculatum Ness; Cinnamomum burmanni (Ness)Blum; Cinnamomum mairei Levl; Cinnamomum chingii Metealf; Cinnamomum chekiangense Nakai.; Cinnamomum camphora; Laurus camphora (L.); Camphora officinarum.; Laurus camphora.; Laurus camphora; Camphora camphora.

“Jinyingzi,” as used herein, refers to extracts of jinyingzi. It is also known as fructus rosae laevigatae and rosa laevigata Michx. A member of the botanical group Rosa is a substance that is capable of providing a similar physiological effect(s) as that provided by jinyingzi in the compositions of the invention, and is preferably selected from a group comprising Fructus rosae laevigatae; Rosa laevigata Michx; Rosa laevigata; Rosa bella Rehd et Wils; Rosa davidii Crep; Rosa macrophylla Lindl.; Rosa amygdalifolia; Rosa cherokeansis; Rosa cherokeensis; Rosa cucumerina Rosa hystrix; Rosa laevigana; Rosa sinica Ait.; Rosa temate; Rosa trifoliate.

“Tougucao,” as used herein, refers to extracts of tougucao. It is also known as speranskia tuberculata (Bge.) Baill. A member of the botanical group Speranskia is a substance that is capable of providing a similar physiological effect(s) as that provided by tougucao in the compositions of the invention, and is preferably selected from a group comprising Speranskia tuberculata (bunge) Ball.; Speranskia tubescens Mak.; Impatiens balsamina L; Incarvillea sinensis Lari; Vicia amoena Fisch; Gaultheria yunnanensis (Franch.) Rehd; Clematis intricate Bunge; Clematis glauca Wall var. angustifolia Ledeb.; Clematis aethusaefolia Turcz; Cynanchum paniculatum (Bunge)Kitag; Vicia cracca L.; Vicia pseudo-orobus Fisch.et Mey; Thesium chinense Turcz.

“Xixiancao,” as used herein, refers to extracts of xixiancao. It is also known as Siegesbeckia pubescens Mak. A member of the botanical group Siegesbeckia is a substance that is capable of providing a similar physiological effect(s) as that provided by xixiancao in the compositions of the invention, and is preferably selected from a group comprising Siegesbeckia pubescens Mak; Siegesbeckia pubescens Makino; Siegesbeckia pubescens ((Makino.)Makino.); Siegesbeckia orientalis L.; Siegesbeckia orientalis L. var. pubescens Makino; Siegesbeckia orientalis pubescens; Siegesbeckia glabrescens Mak.; Siegesbeckia glabrescens Makino.

“Duhuo,” as used herein, refers to extracts of duhuo. It is also known as radix angelicae pubescentis and angelica pubescens Maxim. A member of the botanical group Angelica pubescens is a substance that is capable of providing a similar physiological effect(s) as that provided by duhuo in the compositions of the invention, and is preferably selected from a group comprising Radix angelicae pubescentis; Angelica pubescens Mak.; Angelica pubescens Maxim.; Angelica pubescens; Angelica dahurica Benth et Hook; Heracleum hemsleyanum Diels; Heracleum lanatum Michx; Aralia cordata Thunb.

“Weilingxian,” as used herein, refers to extracts of weilingxian. It is also known as radix clematidis and clematis chinensis osbeck. A member of the botanical group Clematis is a substance that is capable of providing a similar physiological effect(s) as that provided by weilingxian in the compositions of the invention, and is preferably selected from a group comprising Radix clematidis chinensis; Radix clematidis uncinatae; Radix clematidis manshurica; Radix clematidis hexapetalae; Clematis chinensis osbeck C.; Clematis hexapetale Pall; Clematis uncinata Champ; Clematis manshurica Rupr; Clematis angustifolia Jacq.; Clematis finetiana Levl. Et. Vant; Clematis povoliniana Pamp. Clematis.

“Shenjincao,” as used herein, refers to extracts of shenjincao. It is also known as lycopodium clavatum. A member of the botanical group Lycopodium is a substance that is capable of providing a similar physiological effect(s) as that provided by shenjincao in the compositions of the invention, and is preferably selected from a group comprising Lycopodium clavatum L.; Lycopodium clavatum; Lycopodium clavatum var. laurentianum; Lycopodium clavatum var. subremotum; Lycopodium clavatum var. tristachyum; Lycopodium cemnum L.; Lycopodium obscurum L. Muscus clavatum; Muscus terrestris.

“Vitamin B-1,” as used herein, refers to the thiamine member of the vitamin B series or complex.

“Vitamin B-2,” as used herein, refers to the riboflavin member of the Vitamin B series or complex.

“Vitamin B-6,” as used herein, refers to the pyridoxine member of the Vitamin B series or complex.

“Vitamin B-12,” as used herein, refers to the cobalamin member of the Vitamin B series or complex.

“Vitamin C,” as used herein, refers to ascorbic acid and salts thereof.

“Vitamin E,” as used herein, refers to D alpha-tocopherol, preferably in its succinate form.

“Devil's claw,” as used herein, refers to extracts of devil's claw. It is also known as Harpagophytum procumbens. A member of the botanical group Harpagophytum is a substance that is capable of providing a similar physiological effect(s) as that provided by devil's claw in the compositions of the invention, and is preferably selected from a group comprising Harpagophytum procumbens.

“Black cohosh,” as used herein, refers to extracts of black cohosh. It is also known as rhizome of Cimicifuga racemosa and Cimicifuga racemosa. A member of the botanical group Cimicifuga is a substance that is capable of providing a similar physiological effect(s) as that provided by black cohosh in the compositions of the invention, and is preferably selected from a group comprising Cimicifuga racemosa; Cimicifuga cordifolia; Cimicifuga racemosa var. cordifolia; Cimicifuga heracleifolia Komar.; Cimicifuga dahurica (Turcz) Maxim; Ciimicifuga foetida L.; Actaea racemosa.

“Selenium,” as used herein, refers to the form(s) of the mineral known to persons of skill in the art to be therapeutically effective in the body of the individual. It is preferably provided as selenium aminoate.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30° C. to 100° C., most preferably 45° C. to 75° C.). The extract is preferably treated to yield a form suitable for mixing of two or more substances. The form is preferably a dried powder. The powder form is yielded from preferably at least about a 1:10, more preferably at least about a 1:8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in therapeutic effectiveness to those produced by the preceding preferred extraction method can be used for the purposes of this invention.

Formulation of the Compositions

Each substance contained in the compositions provided by this invention is provided in an amount that lies within specific quantitative ranges herein disclosed to be effective for treating back or leg discomfort.

According to the present invention, an effective amount of a composition comprises preferably from 20 mg to 400 mg, more preferably 80 mg to 300 mg, most preferably 120 mg to 200 mg of a member of the botanical group Eucommia (such as duzhong); preferably 20 mg to 400 mg, more preferably 80 mg to 300 mg, most preferably 120 mg to 200 mg of a member of the botanical group Achyranthes (such as huainiuxi); preferably 100 mg to 1250 mg, more preferably 300 mg to 1000 mg, most preferably 600 mg to 800 mg of a member of the botanical group Millettia (such as jixueteng); preferably 20 mg to 400 mg, more preferably 80 mg to 300 mg, most preferably 120 mg to 200 mg of a member of the botanical group Homalomena (such as qiannianjian); preferably 30 mg to 600 mg, more preferably 100 mg to 450 mg, most preferably 200 mg to 300 mg of a member of the botanical group Cinnamomum cassia (such as rougui); and/or preferably 100 mg to 1500 mg, more preferably 400 mg to 1200 mg, most preferably 700 mg to 900 mg of a member of the botanical group Siegesbeckia (such as xiquancao).

In the various embodiments of the invention, the compositions further comprise preferably from 20 mg to 400 mg, more preferably 80 mg to 300 mg, most preferably 120 mg to 200 mg of a member of the botanical group Rosa (such as jinyingzi); preferably 20 mg to 400 mg, more preferably 80 mg to 300 mg, most preferably 120 mg to 200 mg of a member of the botanical group Speranskia (such as tougucao); preferably 80 mg to 1200 mg, more preferably 200 mg to 1000 mg, most preferably 350 mg to 750 mg of a member of the botanical group Angelica pubescens (such as duhuo); preferably 50 mg to 800 mg, more preferably 150 mg to 650 mg, most preferably 300 mg to 500 mg of a member of the botanical group Clematis (such as weilingxian); preferably 10 mg to 250 mg, more preferably 50 mg to 200 mg, most preferably 80 mg to 140 mg of a member of the botanical group Lycopodium (such as shenjincao); preferably 2.5 mg to 75 mg, more preferably 10 mg to 65 mg, most preferably 20 mg to 40 mg vitamin B-1; preferably 2.5 mg to 75 mg, more preferably 10 mg to 65 mg, most preferably 20 mg to 40 mg vitamin B-2; preferably 10 mg to 200 mg, more preferably 20 mg to 150 mg, most preferably 50 mg to 100 mg vitamin B-6; preferably 0.01 mg to 0.2 mg, more preferably 0.05 mg to 0.15 mg, most preferably 0.1 mg to 0.12 mg vitamin B-12; preferably 15 mg to 450 mg, more preferably 75 mg to 350 mg, most preferably 125 mg to 250 mg vitamin C; preferably 15 mg to 250 mg, more preferably 50 mg to 175 mg, most preferably 100 mg to 125 mg vitamin E; preferably 5 mg to 150 mg, more preferably 25 mg to 100 mg, most preferably 50 mg to 75 mg of a member of the botanical group Harpagophytum (such as devil's claw); preferably 5 mg to 150 mg, more preferably 25 mg to 100 mg, most preferably 50 mg to 75 mg of a member of the botanical group Cimicifuga (such as black cohosh); and/or preferably 10 mg to 250 mg, more preferably 50 mg to 200 mg, most preferably 80 mg to 140 mg selenium.

Selection of suitable members of a particular botanical group to be included in a composition can be achieved using methods known in the art. For example, a suitable member of the botanical group Eucommia would be expected to be capable of providing a similar physiological effect(s) as that provided by duzhong in a composition of the invention. Such a member can be selected based on, for example, whether it is known, shown and/or suspected to possess said similar physiological effect(s). Thus, for example, a determination of whether a candidate substance can be a member of the botanical group Eucommia can be done based on, for example, a similar pharmacological or medicinal classification for both the candidate substance and duzhong. However, the activity and/or function provided by a particular substance, such as duzhong, need not be identified or specified. A determination of whether a candidate substance can be a member of a particular botanical group, for example the Eucommia group, can also be empirical, for example, by substituting said candidate substance for duzhong in a composition, and assessing the relevant therapeutic effect(s) of the composition. Such a determination can be done using methods and techniques known in the art.

According to this invention, the compositions can be formulated in whatever form that retains the efficacy of the composition for treating back or leg discomfort. Preferably, the compositions are packaged in the form of capsules. The capsules are preferably of size “0”, “00”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington 's Pharmaceutical Sciences, 18th Edition, Mack Publishing (1990).

Administration of Compositions

Compositions in any of the physical forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. Administration of an effective amount is preferably completed within 24 hours. A composition can be ingested alone, or with any other substance, such as a liquid, that aids ingestion of the compositions. Ingestion of the compositions can be before or after food consumption.

### EXAMPLES

### Example 1

An Illustrative Example of the Formulation of A Single “00” Capsule, and the Production Thereof

A composition shown to be effective for treating back or leg discomfort contained substances in the indicated quantities as listed in Table 1.

TABLE 1

| SUBSTANCE [commercial source] | AMOUNT (mg) |
|---|---|
| Duzhong [Mayway, Oakland, CA] | 33 |
| Huainiuxi [Qualiherb, Cerritos, CA] | 33 |
| Jixueteng [Qualiherb, Cerritos, CA] | 117 |
| Qiannianjian [Qualiherb, Cerritos, CA] | 33 |
| Rougui [Qualiherb, Cerritos, CA] | 50 |
| Xiquancao [Qualiherb, Cerritos, CA] | 133 |
| Jinyingzi [Mayway, Oakland, CA] | 33 |
| Tougucao [Qualiherb, Cerritos, CA] | 33 |
| Duhuo [Qualiherb, Cerritos, CA] | 100 |
| Weilingxian [Mayway, Oakland, CA] | 67 |
| Shenjincao [Qualiherb, Cerritos, CA] | 17 |
| Vitamin B-1 [Acta, Sunnyvale, CA] | 5 |
| Vitamin B-2 [Acta, Sunnyvale, CA] | 5 |
| Vitamin B-6 [Acta, Sunnyvale, CA] | 15 |
| Vitamin B-12 [Acta, Sunnyvale, CA] | 0.0125 |
| Vitamin C (ascorbic acid) [Acta, | 30 |
| Sunnyvale, CA] | |
| Vitamin E (D Alpha-Tocopherol) [Acta, | 21.19 |
| Sunnyvale, CA] | |
| Devil's Claw [Acta, Sunnyvale, CA] | 10 |
| Black cohosh [Acta, Sunnyvale, CA] | 10 |
| Selenium [Acta, Sunnyvale, CA] | 20 |

Capsules containing the composition above were manufactured according to the method used by the commercial manufacturer, Acta (Sunnyvale, Calif.). Briefly, the substances listed above, in powder form and obtained from the commercial sources indicated, were mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 706 mg of the filtered mixture was then packaged into each size “00” capsule.

### Example 2

A First Illustrative Example of Treatment of Back or Leg Discomfort

10 individuals complaining of back pain were asked to rate the severity of their symptom on a scale of 1 to 10, 1 being normal condition and 10 being severe pain. The individual were instructed to assess frequency of pain (e.g., occasional, intermittent, or constant) as a primary criterion for pain severity. The composition of Example 1 was then administered in conjunction with an enhancing composition (which by itself is not efficacious in treating back or leg discomfort conditions), which is disclosed in co-pending application entitled “Compositions And Methods For Enhancing Therapeutic Effects” (U.S. Provisional Application Ser. No. 60/208,990; U.S. patent application Ser. No. ______, filed even date herewith), which is hereby incorporated in its entirety by reference. Each capsule containing the enhancing composition contained substances described in Table 2 below.

TABLE 2

| SUBSTANCE [Commercial Source] | AMOUNT (mg) |
|---|---|
| Xuejie [Min Tong Herb, Oakland, CA] | 133 |
| Yanhusuo [Mayway, Oakland, CA] | 83 |
| Baishaoyao [Qualiherb, Cerritos, CA] | 83 |
| Shanqi [Sun Ten, Irvine, CA] | 250 |
| Gancao [Qualiherb, Cerritos, CA] | 83 |
| White willow bark [Acta, Sunnyvale, | 10 |
| CA] | |
| Black cohosh root [Acta, Sunnyvale, | 10 |
| CA] | |
| L-carnitine [Acta, Sunnyvale, CA] | 50 |
| Vitamin E (D Alpha-Tocopherol | 21.19 |
| succinate) [Acta, Sunnyvale, CA] | |
| Vitamin C [Acta, Sunnyvale, CA] | 30 |

Individuals were administered various numbers of capsules (as indicated in the data table below) of the composition of Example 1 and the enhancing composition of Table 2. The individual was asked to assess her symptom daily using the 0 to 10 scale for the duration of the study. A therapeutic effect is obtained if there is a decrease in the severity of the symptom.

The results for treatment of back pain are described in Table 3 below.

TABLE 3

| | Before | | | | |
|---|---|---|---|---|---|
| | treatment | 1-3 days | 4-6 days | 7-10 days | 2 weeks |
| Backpain | 9 | 4 | 3 | 0 | 0 |
| (symptom | | | | | |
| score) | | | | | |
| B & L (average | | 9 | 6 | 3 | 0 |
| capsules/day) | | | | | |
| Enh. (average | | 9 | 6 | 3 | 0 |
| capsules/day) | | | | | |

B & L: Composition of Example 1
Enh.: Composition of Table 2

The results indicate a significant therapeutic effect with respect to back pain in individuals who were administered the composition of Example 1.

### Example 3

A Second Illustrative Example of Treatment of Back or Leg Discomfort

A randomized, double-blinded and controlled study is performed. Control groups use either an existing treatment (such as nonsteroidal anti-inflammatory drugs or aspirin) or a sugar pill placebo. Dosage amount and schedule are 1 to 6 capsules (of the composition of Example 1) daily. At least 20 individuals are tested, randomly assigned in approximately equal number to treatment (administered the composition of Example 1) and control groups. Individuals are evaluated to suffer from a particular back or leg discomfort symptom, such as pain and/or soreness of the back and/or leg.

The study is carried out for at least 1 week. During the treatment course, a dosage amount selected from the range of 1 to 6 capsules is administered to each individual once or multiple times daily, not exceeding 6 capsules per day. Capsules are administered before or after food consumption.

A clinical questionnaire is used to evaluate individuals' back or leg symptoms. A clinical coordinator and/or physician evaluates the individual's back or leg discomfort symptoms and fills out the questionnaire. Frequency of pain/soreness (e.g., occasional, intermittent, or constant) is used as a primary criterion for assessing pain/soreness severity. Evaluations can be performed daily, or more or less frequently depending on statistical or clinical (ability to detect or track symptomatic improvements) need.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales); and (4) non-efficacy, as defined as no improvement in symptoms.

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

1. A composition for treating back or leg discomfort comprising 2 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

2. A composition for treating back or leg discomfort comprising 3 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

3. A composition for treating back or leg discomfort comprising 4 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

4. A composition for treating back or leg discomfort comprising 5 substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

5. A composition for treating back or leg discomfort comprising a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

6. A composition selected from the group consisting of the compositions of claims 1 to 5, further comprising a substance selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

7. A composition selected from the group consisting of the compositions of claims 1 to 5, further comprising 2 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

8. A composition selected from the group consisting of the compositions of claims 1 to 5, further comprising 3 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

9. A composition selected from the group consisting of the compositions of claims 1 to 5, further comprising 4 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

10. A composition selected from the group consisting of the compositions of claims 1 to 5, further comprising 5 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

11. A composition selected from the group consisting of the compositions of claims 1 to 5, further comprising 6 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

12. A composition selected from the group consisting of the compositions of claims 1 to 5, further comprising 7 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

13. A composition selected from the group consisting of the compositions of claims 1 to 5, further comprising 8 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

14. A composition selected from the group consisting of the compositions of claims 1 to 5, further comprising 9 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

15. A composition selected from the group consisting of the compositions of claims 1 to 5, further comprising 10 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

16. A composition selected from the group consisting of the compositions of claims 1 to 5, further comprising 11 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

17. A composition selected from the group consisting of the compositions of claims 1 to 5, further comprising 12 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

18. A composition selected from the group consisting of the compositions of claims 1 to 5, further comprising 13 substances selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

19. A composition selected from the group consisting of the compositions of claims 1 to 5, further comprising a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.

20. The composition of any of claims 1-5, wherein the member of the botanical group Eucommia is duzhong, the member of the botanical group Achyranthes is huainiuxi, the member of the botanical group Millettia is jixueteng, the member of the botanical group Homalomena is qiannianjian, the member of the botanical group Cinnamomum cassia is rougui and the member of the botanical group Siegesbeckia is xiquancao.

21. The composition of claim 6, wherein the member of the botanical group Rosa is jinyingzi, the member of the botanical group Speranskia is tougucao, the member of the botanical group Angelica pubescens is duhuo, the member of the botanical group Clematis is weilingxian, the member of the botanical group Lycopodium is shenjincao, the member of the botanical group Harpagophytum is devil's claw, and the member of the botanical group Cimicifuga is black cohosh.

22. A method of treating back or leg discomfort comprising administering to the individual an effective amount of a composition selected from the group consisting of the compositions of claims 1 to 5.

23. A method of making a composition for treating back or leg discomfort, said method comprising combining at least two substances selected from the group consisting of a member of the botanical group Eucommia, a member of the botanical group Achyranthes, a member of the botanical group Millettia, a member of the botanical group Homalomena, a member of the botanical group Cinnamomum cassia and a member of the botanical group Siegesbeckia.

24. The method of claim 23, wherein said at least two substances are combined with at least one substance selected from the group consisting of a member of the botanical group Rosa, a member of the botanical group Speranskia, a member of the botanical group Angelica pubescens, a member of the botanical group Clematis, a member of the botanical group Lycopodium, vitamin B-1, vitamin B-2, vitamin B-6, vitamin B-12, vitamin C, vitamin E, a member of the botanical group Harpagophytum, a member of the botanical group Cimicifuga and selenium.
